(19) United States
(12) Patent Application Publication
(10) Pub. No.: US 2024/0217894 A1
(43) Pub. Date: Jul. 4, 2024

(54) METHOD FOR CONVERTING CO2 INTO METHANE

(71) Applicant: ORANO CHIMIE-ENRICHISSEMENT, Chatillon (FR)

(72) Inventors: Lai Truong-Phuoc, Strasbourg (FR); Jean-Mario Nhut, Plobsheim (FR); Giuliano Giambastiani, Florence (IT); Cuong Pham-Huu, Strasbourg (FR); Alex Jourdan, Orange (FR)

(21) Appl. No.: 18/557,410

(22) PCT Filed: Apr. 21, 2022
(86) PCT No.: PCT/FR2022/050745

§ 371 Date: Oct. 26, 2023

(30) Foreign Application Priority Data
Apr. 29, 2021 (FR) 2104508

(51) Int. Cl. C07C 1/12; B01J 23/83; B01J 37/02; B01J 37/08

(52) U.S. Cl. CPC C07C 1/12; B01J 23/83; B01J 37/0201; B01J 37/088

(57) ABSTRACT

A method for converting CO₂ into methane wherein hydrogen is contacted with a gas feed including CO₂ in at least one methanation reactor comprising a catalyst bed at a temperature in the catalytic bed comprised between 160° C. and 550° C., at a pressure comprised between 0.1 MPa and 1 MPa, with a gas hourly space velocity comprised between 10 m³/kg/h and 50 m³/kg/h and with an H₂/CO₂ molar ratio comprised between 1 and 8 and wherein the catalyst contains Ni metal deposited on a uranium oxide support of formula UO₂₊ₓ, with x being comprised between 0.01 and 0.6, and the nickel mass content of between 5% and 40% of nickel metal relative to the total mass of the catalyst.

The present invention relates to a method for the catalytic conversion of carbon dioxide in the presence of hydrogen into methane (methanation method) using a nickel-based catalyst dispersed on a support based on uranium oxide.

### PRIOR ART

Sustainable energy production, combined with moderate consumption practices, represents a challenge for our civilisation. Over the last decade, the continued increase in global energy demand and collective awareness of the problem of global warming have led to the development of means for producing electrical energy from renewable sources.

This massive integration of renewable energy sources into the energy landscape, however, comes up against the problem of managing electricity networks. The intermittent and localised nature of most of these sources, particularly wind and solar sources, complicates the balancing of these networks at any time, between production and demand for electrical energy. Added to this is the need to be able to manage the storage of surplus electricity so as to be able to easily benefit therefrom in the event of a drop in the efficiency of renewable energy production means.

Current storage means only allow limited amounts of energy to be stored and can therefore only meet needs that extend over short periods of time, a few days at most.

In recent years, the idea of an alternative storage means to conventional technologies has emerged and proposes using energy in chemical form. This storage means, called Power-to-Gas, thus converts electrical energy into gas, for example hydrogen or methane, which is used as a storage vector. This chemical storage has the advantage that it can be maintained over long periods compared to those of the systems currently implemented.

The conversion of electrical energy into gas moreover offers numerous possibilities for the end use of this energy, such as domestic heating, industrial use or else personal mobility.

Power-to-Gas method consists in particular of implementing one or two conversion steps depending on the storage gas chosen. This vector can be either hydrogen, produced by the electrolysis of water power supplied by solar or wind energy, or methane, produced in a second step called methanation step which carries out the conversion of hydrogen and carbon dioxide into methane. In addition to allowing the recovery of CO₂ considered as a by-product of industry, carrying out this additional step allows to limit the constraints and the cost of adapting storage, distribution and end use of the energy vector.

The methanation reaction converts CO₂ in the presence of hydrogen into methane and water (Equation 1).

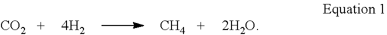

This reaction is strongly exothermic and releases significant heat (ΔH°_{298K}=−165.0 KJ/mol). According to Le Chatelier's principle, the formation of methane is favoured at low temperature and under high pressure. However, the CO₂ molecule is a stable linear molecule, composed of two O═C double bonds, hence the need to provide excess energy to activate this molecule and to use a catalyst to overcome the significant kinetic limitations of this reaction.

Numerous research carried out to develop catalysts allowing to reduce the energy barrier for activation of this reaction have shown that catalysts based on noble metals are particularly active but their high prices constitute a limit to their uses. It appears that catalysts based on Ni dispersed on a support are the most promising for the methanisation of COₓ (x=1, 2) due to their good catalytic performance and their relatively low price. Commonly used nickel catalyst supports are oxides such as Al₂O₃, SiO₂, TiO₂, ZrO₂ and CeO₂. Among them, Ni/SiO₂ and Ni/Al₂O₃ have been widely studied because of their good initial activities but suffer, when used at high temperatures, from deactivation phenomena due to sintering of the particles of the active phase (reducing the number of active sites) and significant carbon deposits (coke) thus blocking the access of the reagents to the active sites. Currently catalysts based on Ni supported on an Al₂O₃ support are available from manufacturers such as Johnson Matthey, Haldor-Topsøe or Clariant-Süd Chemie.

The publication of Berry and al. (Applied Catalysis A: General 100 (1993) 131-143) is also know in the prior art which focuses on catalysts based on Ni and uranium oxide useful for the CO₂ methanation reaction. The catalysts are prepared by evaporating an aqueous solution containing nickel nitrate and uranyl nitrate until a viscous residue is formed which solidifies at room temperature. The solid thus obtained is then calcined in air at a temperature of 1000° C.

A purpose of the present invention is to propose a CO₂ methanation method which meets several criteria, in particular in terms of CO₂ conversion rate, CH₄ selectivity, productivity and which can be operated in particular at temperatures below 350° C. and, preferably, below 300°C., or even below 260° C.

### SUMMARY OF THE INVENTION

The invention therefore relates to a method for converting CO₂ into methane wherein:
hydrogen is contacted with a gas feed comprising CO₂ in at least one methanation reactor comprising a catalyst bed at a temperature in the catalytic bed comprised between 160° C. and 550° C., at a pressure comprised between 0.1 MPa and 1 MPa, with a gas hourly space velocity comprised between 10 m^{3/}kg/h and 50 m³/kg/h and with an H₂/CO₂ molar ratio comprised between 1 and 8;
the catalyst contains Ni metal deposited on a uranium oxide support of formula UO₂₊ₓ, with x being comprised between 0.01 and 0.6, and the nickel mass content of which is comprised between 5% and 40% of nickel metal relative to the total mass of the catalyst; and wherein:
the catalyst is prepared by a method comprising the following steps a) to c):
   a) a support precursor consisting essentially of a uranium (IV) and/or uranium (VI) oxide is impregnated with a solution containing a nickel precursor and a polar solvent;
   b) the impregnated support precursor is calcined in air and at a temperature of at least 250° C.; and
   c) the impregnated and calcined support precursor is reduced under hydrogen at a temperature of at least 300° C.

Surprisingly, the applicant has found that the use of a catalyst comprising nickel metal dispersed on a support based on uranium oxide UO₂₊ₓ with x being comprised between 0.01 and 0.6 has an exacerbated activity for the conversion of CO₂ into methane, so that the method can be carried out at lower temperatures than that of the prior art, while maintaining a high yield and selectivity in CH₄, that is to say respectively greater than 60% and close to 100%.

The term “consisting essentially of a uranium (IV) and/or uranium (VI) oxide” is understood to mean a support the uranium (IV) and/or uranium (VI) oxide content of which is at least 90% by mass.

The temperature of the catalytic bed can be measured by any method known to the person skilled in the art, for example by means of one or more thermocouples disposed in said bed or by laser pyrometry.

According to particular embodiments, the method comprises one or more of the following features, taken in isolation or in all technically possible combinations.

The method for preparing the catalyst may comprise, before the calcination step b), a step wherein the impregnated support precursor is dried at a temperature below 200° C. in order to eliminate in particular the solvent from the impregnation solution. Preferably, the solution contains a polar solvent wherein the nickel precursor is solubilised. Preferably, it is an aqueous solution containing the nickel precursor.

Preferably, the calcination step b) is carried out at a temperature of at least 300° C. for at least 1 hour, preferably for at least 2 hours.

Preferably, the reduction step c) is carried out at a temperature of at least 350° C. for at least 1 hour, preferably for at least 2 hours. The hydrogen necessary for the reduction is provided in the form of pure hydrogen. This reduction step not only converts uranium oxide (VI) or uranium (IV) and (VI) oxide (for example U₃O₈) into UO₂₊ₓ and forms nickel metal.

The support precursor based on uranium (IV) and/or uranium (VI) oxide can be selected from UO₂, UO₃, UO₄ and U₃O₈. Preferably, the support precursor is U₃O₈.

According to the invention, the support precursor can be in any form. For example, the support precursor has a morphology in the form of cylindrical or multilobed extrudates, spheres or a powder of variable particle size. According to one embodiment, when the precursor of the support is a powder, it can advantageously be shaped (for example beads or extrudates) after the step of impregnating the nickel precursor.

For the impregnation step, the nickel precursor can be selected from nickel hydroxide, hydroxycarbonate, carbonate and nitrate, with a preference for nickel nitrate. Step a) of impregnating the nickel precursor can be carried out using dry or excess impregnation methods.

Step c) can be carried out ex situ or in situ, that is to say directly in the methanation reactor.

To carry out the conversion method, the hydrogen and the CO₂ are sent separately into the methanation reactor, for example in a downward direction. Alternatively, the two gas reagents are mixed beforehand before being sent to the methanation reactor.

The methanation reactor is an adiabatic, isothermal or hybrid type reactor. Preferably, the methanation reactor is an adiabatic reactor with a fixed or fluidised catalyst bed. Heating the reactor can be carried out by any method known to the person skilled in the art, for example by means of a resistance disposed in the catalytic bed, of an internal (serpentine type) or external heat exchanger system.

According to one embodiment, when the catalyst bed is a fixed bed, the catalytic bed is subjected to an alternating electromagnetic field so as to heat the catalytic bed by induction.

In order to promote induction heating, the catalyst bed advantageously comprises a susceptor, that is to say an element which, when subjected to an alternating electromagnetic field, is capable of converting electromagnetic energy into heat and to communicate it to the catalyst. This may be the result of hysteresis losses and/or eddy currents induced in the susceptor which depend in particular on the electrical and magnetic properties of the susceptor material. Hysteresis losses occur in ferromagnetic or ferrimagnetic susceptors and result from the switching of magnetic domains within the material when the latter is subjected to the influence of an alternating electromagnetic field. Eddy currents can be induced if the susceptor is electrically conductive. In the case of an electrically conductive ferromagnetic or ferrimagnetic susceptor, heat can be generated by both eddy currents and hysteresis losses. In this case, the heating is carried out essentially on the surface of the susceptor which can transmit the heat to the surface of the catalyst with which it is in contact. For example, the susceptor can be selected from carbonaceous/graphitic materials, metals or metal alloys which are not reactive for the targeted reaction such as, for example, aluminium, iron, copper, bronze, stainless steel, ferritic stainless steel, martensitic stainless steel and austenitic stainless steel. The susceptor can be either in direct contact with the catalyst or separated from the catalyst by a non-thermally insulating wall so as to allow rapid and homogeneous transfer of heat to the catalyst.

Advantageously, in order to address the challenges of the energy transition, the method according to the invention is carried out with a renewable energy source in order to store the latter in chemical form.

According to one embodiment, the conversion method uses a reactor with a fixed catalyst bed and wherein the gas hourly space velocity is fixed at a value of at least 15 m³/kg/h so as to maintain a temperature in the catalytic bed at least at 200° C., whereby the conversion reaction is carried out without the addition of external heat.

The invention also relates to a CO₂ methanation catalyst comprising nickel metal deposited on a uranium oxide support of formula UO₂₊ₓ with x being comprised between 0.01 and 0.6 and wherein the mass content of nickel metal is comprised between 5% and 40% of Ni relative to the total mass of the catalyst. Preferably, the catalyst according to the invention consists of nickel metal deposited on a uranium oxide support of formula UO₂₊ₓ with x being comprised between 0.01 and 0.6.

The nickel metal content is preferably comprised between 10% and 20% by mass of nickel relative to the total mass of catalyst.

Finally, the invention relates to a method for preparing a CO₂ methanation catalyst comprising nickel metal deposited on a uranium oxide support of formula UO₂₊ₓ with x being comprised between 0.01 and 0.6 and wherein the mass content of nickel metal is comprised between 5% and 40% of Ni relative to the total mass of the catalyst, which method comprises the following steps a) to c):
a) a support precursor consisting essentially of a uranium (IV) and/or uranium (VI) oxide is impregnated with a solution containing a nickel precursor and a polar solvent;
b) the impregnated support precursor is calcined in air and at a temperature of at least 250° C.;
c) the impregnated and calcined support precursor is reduced under hydrogen at a temperature of at least 300° C.

### DETAILED DESCRIPTION OF THE INVENTION

Description of gas feeds

The method according to the invention allows to treat a gas feed having a CO₂ volume content greater than 30%, preferably greater than 50% and even better greater than 90%. Alternatively, the gas feed containing CO₂ may be mixed with methane which may have a volume content of at most 50%.

This gas CO₂ feed is, for example, a gas effluent from a biomass methanisation unit, a gasification unit, an oil refining unit or a cement plant. The CO₂ can also come from CO₂ capture units.

As for the gas hydrogen charge (H₂), it can be obtained from the electrolysis of water or come from a catalytic reforming unit for heavy petroleum cuts.

According to a preferred embodiment which is part of the concept of Power-to-Gas methods allowing to store renewable energies in chemical form, hydrogen is produced in water electrolysis units powered by solar or wind power plants.

Preferably, the H₂ volume content of this filler is greater than 90% and, preferably, greater than 95%.

According to the invention, the CO₂ and dihydrogen feeds can be mixed before being sent to the methanation reactor (preferred mode of operation) or else be distributed separately in the methanation reactor. The conversion reaction is carried out in the presence of a gas mixture whose H₂/CO₂ molar ratio is comprised between 1 and 8, preferably comprised between 1 and 4, and more preferably equal to 4.

Description of the methanation catalyst according to the invention

The methanation method according to the invention uses a heterogeneous catalyst comprising metallic nickel supported on a uranium oxide corresponding to the formula UO₂₊ₓ with x being comprised between 0.01 and 0.6.

Nickel is present in the catalyst at a mass content comprised between 5% and 40% of Ni metal relative to the total mass of the catalyst. Preferably, the mass content of Ni metal is comprised between 10% and 20% relative to the total mass of the catalyst.

The catalyst according to the invention differs from that of the prior art described in Applied Catalysis A: General 100 (1993) 131-143, in that it is obtained in particular by impregnation of a precursor of the support with a solution containing a nickel salt, then calcination of the impregnated support and finally reduction under hydrogen of the product of calcination. Advantageously, in particular in order to reduce the calcination treatment time, the impregnated support is subjected to a drying step before the calcination step.

In the context of the invention, the support precursor based on uranium oxide can come from the nuclear sector for enriching natural uranium in uranium 235 (U²³⁵), which provides uranium called “depleted uranium”, that is to say whose U²³⁵ mass content is less than 0.7%, generally comprised between 0.2% and 0.4%.

In a first step of the catalyst synthesis method, the uranium oxide-based support precursor is impregnated with a solution containing a soluble nickel salt. The solution contains a polar solvent, which is preferably water, wherein a nickel salt which can be selected from nickel hydroxide, hydroxycarbonate, carbonate and nitrate is dissolved. The nickel content of the impregnation solution can take any value but, preferably, this value is less than the saturation of the salt in the solvent used. Preferably, use is made of a solution most concentrated in soluble nickel salt while avoiding saturation of the impregnation solution. In the case where the catalyst according to the invention contains high Ni contents (for example greater than 20%), it is possible to proceed by successive impregnations of the support with optionally intermediate drying and calcination steps.

The precursor of the support can be found in the form of small diameter, cylindrical or multilobed extrudates (trilobes, quadrilobes, etc.), spheres, rings, monoliths in a honeycomb structure or in the form of a powder.

The BET specific surface area of the precursor is generally comprised between 1 m²/g and 10 m²/g, preferably between 1 m²/g and 5 m²/g. The specific surface area is determined by nitrogen porosimetry.

The step of contacting said support precursor with an impregnation solution containing nickel can be carried out either by slurry impregnation, or by excess impregnation, or by dry impregnation, or by any other means known to the person skilled in the art. Impregnation at equilibrium (or in excess) consists of immersing the support in a volume of solution (often significantly) greater than the porous volume of the support while maintaining the system under agitation to improve the exchanges between the solution and the support or catalyst. An equilibrium is finally reached after diffusion of the different species in the pores of the support. Control of the amount of elements deposited is ensured, for example, by the prior measurement of an adsorption isotherm which allows to relate the concentration of the elements to be deposited contained in the solution to the amount of elements deposited on the solid in equilibrium with this solution.

Dry impregnation consists, in turn, in introducing a volume of impregnation solution equal to the porous volume of the support. Dry impregnation allows all the additives contained in the impregnation solution to be deposited on a given support or catalyst.

The nickel solution impregnation step can advantageously be carried out by one or more impregnations in excess of solution or, preferably, by one or more dry impregnations.

This impregnation step can be carried out at a temperature comprised between 18° C. and 50° C., preferably between 20° C. and 30° C.

At the end of step a), the impregnated support can advantageously be allowed to mature so as to allow homogeneous dispersion of the impregnation solution within the support. Any maturation step is advantageously carried out at atmospheric pressure, at a temperature comprised between 18° C. and 50° C. and, preferably, at room temperature. Generally, a maturation time comprised between 10 minutes and 48 hours and, preferably, comprised between 30 minutes and 6 hours, is sufficient.

When the support precursor is a powder, after the step of impregnating the nickel precursor, the latter is advantageously shaped, for example, by spheronisation or by extrusion.

The support precursor after impregnation is optionally subjected to a drying step at a temperature below 200° C., advantageously comprised between 50° C. and 150° C., preferably between 70° C. and 150° C., very preferably between 75° C. and 130° C. The drying step is preferably carried out under an atmosphere containing oxygen, preferably under air. The drying step can be carried out by any technique known to the person skilled in the art. It is advantageously carried out at atmospheric pressure or at reduced pressure. Preferably, this step is carried out at atmospheric pressure. It is advantageously carried out in a crossed bed using air or any other hot gas. Preferably, when the drying is carried out in a fixed bed, the gas used is either air or an inert gas such as argon or nitrogen. Very preferably, the drying is carried out in a crossed bed in air. Preferably, the drying step has a duration comprised between 5 minutes and 15 hours, preferably comprised between 2 hours and 12 hours.

It should be noted that the impregnation, maturation and/or drying steps can be repeated several times in succession until the desired amount of nickel deposited on the support precursor is achieved.

The impregnated support precursor, optionally matured and dried, is then subjected to a calcination step under an oxidising atmosphere, preferably under air or under diluted oxygen and at a temperature of at least 250° C. and, preferably, at least 300° C.

Typically, the impregnated precursor is calcined at a temperature comprised between 300° C. and 500° C. in air and for 1 hour to 5 hours.

The implementation of the methanisation catalyst according to the invention requires that it is then activated in a reduction step in order to convert at least part of the oxidised forms of nickel generated during the calcination step into nickel metal and to reduce uranium (VI) oxide or uranium (IV) and (VI) oxide into UO₂₊ₓ. To this end, the catalyst is contacted with pure or diluted hydrogen at a temperature at least equal to 300° C., preferably comprised between 350° C. and 500° C., for a time of at least one hour and preferably between 2 hours and 5 hours. This activation by reduction can be carried out ex situ in a dedicated reduction reactor or in situ, that is to say directly in the methanation reactor after loading it into said reactor.

The activated catalyst generally has a BET specific surface area, measured by a nitrogen adsorption isotherm, comprised between 1 m²/g and 10 m²/g and, preferably, comprised between 2 m²/g and 6 m²/g.

The catalyst according to the invention has excellent catalytic activity for the methanation of CO₂ which results in a CH4 yield of at least 60%, preferably at least 80% and with a CH₄ selectivity close to 100%.

### Implementation of the Methanation Method According to the Invention

The CO₂ methanation method according to the invention consists in contacting, in a reactor, the gas feed containing mainly CO₂, hydrogen and the catalyst described above.

In the context of the invention, the gas feed containing CO₂ can be mixed with a flow of hydrogen and the mixture is then sent to the reactor containing the catalyst.

Contact with the catalyst can be carried out in an adiabatic, isothermal or hybrid reactor. “Adiabatic reactor” means a reactor which does not exchange heat with the external environment; the heat released by the exothermic methanation reaction is then used to fuel the reaction and the excess heat part is evacuated by the effluents withdrawn from the reactor.

“Isothermal reactor” means a reactor which is cooled by the circulation of a fluid allowing to counterbalance the local release of heat of reaction.

Finally, “hybrid reactor” is understood to designate a reactor combining the two aforementioned features wherein a cooling flow is applied to partially counterbalance the local release of heat from the reaction but wherein there still exists a significant temperature gradient within the reactor.

The gas reagents are contacted with the catalyst according to the invention at a temperature in the catalytic bed comprised between 160° C. and 550° C., preferably comprised between 180° C.and 350° C.

The method according to the invention is operated with a gas hourly space velocity, ratio between the gas flowrate in m³/kg/h, and the mass of catalyst in kg, comprised between 10 m³/kg/h and 50 m³/kg/h, preferably comprised between 15 m³/kg/h and 30 m³/kg/h and with an H₂/CO₂ molar ratio comprised between 1 and 8 (mol/mol), preferably comprised between 1 and 4 (mol/mol), even comprised between 3 and 4 and even better equal to 4.

The gas feed can be sent into the reactor in a downward or upward direction, preferably in a downward direction with withdrawal of the reaction products at the bottom of the reactor.

The catalyst according to the invention can be used in a fixed bed or fluidised bed reactor.

When the reactor is of the fixed bed type, it may comprise a plurality of perforated tubes wherein the catalyst is disposed, possibly with packing elements. Alternatively, the fixed catalytic bed can be delimited by perforated lower and upper plates whose diameters correspond to the internal diameter of the reactor and between which the catalyst is disposed, possibly with packing elements.

The thermal energy necessary to be supplied into the reactor can be supplied by any method known to the person skilled in the art, in particular with an internal (serpentine type) or external heat exchanger system, by Joule effect, by microwave and by inductive heating.

Unexpectedly, it was observed that the catalysts according to the invention are capable of being heated under the action of an alternating electromagnetic field.

According to a preferred embodiment, the methanation reactor uses a fixed catalyst bed which is subjected to an alternating electromagnetic field which causes it to heat up without contact with the energy source. This heating mode allows to provide the energy necessary for the reaction only to the catalyst while the gas reagents entering and leaving the catalytic bed are neither heated nor cooled.

According to the invention, in order to improve the efficiency of induction heating, the catalytic bed can comprise a mixture of catalyst according to the invention with an electrically conductive material (susceptor) whose role is to communicate additional heat to the catalyst. The use of a susceptor is recommended when the catalyst has a mass nickel content less than 15% relative to the total mass of catalyst. For example, the susceptor material can be selected from carbonaceous/graphitic materials, metals or alloys of metals which are not reactive for the targeted reaction such as, for example, aluminium, iron, copper, bronze, stainless steel, ferritic stainless steel, martensitic stainless steel and austenitic stainless steel. According to an alternative embodiment, the susceptor is separated from the catalytic bed through a non-thermally insulating wall allowing the transfer of heat to the catalyst.

This implementation by induction heating has several advantages:
precise adjustment of the temperature within the catalytic bed;
extremely rapid regulation, both up and down, of the temperature in the catalytic bed;
the entering reagent is not heated, thus it contributes to efficiently extracting the thermal energy released by the reaction;
to the extent that only the catalyst is heated and not its immediate environment, the evacuation and maintenance of the temperature in the catalytic bed are favoured, which allows to minimise the risks of thermal runaway;
improved energy efficiency;
the water vapour generated by the reaction can be partly condensed in the empty space between the catalyst grains, because only the solid is heated, and thus promote the conversion of the reagents by reducing competitive adsorption problems on the surface of the catalyst.

To carry out induction heating, the reactor comprises an inductive device capable of generating an electromagnetic field. The inductive device can be disposed inside the reactor so as to surround the catalytic bed so that the magnetic field it generates is essentially perpendicular to the thickness of the catalytic bed. According to a second embodiment, the inductive device is disposed at the catalytic bed but outside or in the wall of the reactor. This second embodiment has the advantage that the inductor is decoupled from the chemical environment and thus allows easier control of the inductor. However, in this second embodiment, preference will be given to the use of a reactor made of an electrically non-conductive material such as, for example, glass or ceramic. The assembly can be protected by another external enclosure.

The inductive device is, for example and in a non-limiting manner, a helical induction coil extending over the thickness of the fixed catalytic bed or else a part forming a ring whose height corresponds substantially to the thickness of the catalytic bed.

It should be noted that the catalyst according to the invention is particularly suitable for this type of induction heating because it has a high density, of the order of 6 g/cm³ to 12 g/cm³ and a relatively low specific surface area, of the order of 2 m²/g to 6 m²/g.

Another advantage provided by induction heating is to allow easy separation of the water formed during the conversion of CO₂ without having to resort to a dedicated condensing unit using a heat exchanger system. Indeed, to the extent that only the catalytic bed is subjected to heating, the gas effluent containing a mixture of methane and water undergoes sudden cooling at the outlet of the reactor (“quench” phenomenon) then causing at least partial condensation of the water in the liquid state. The effluent which is withdrawn from the reactor can be sent into a separation flask in order to separate a gas phase containing methane mixed with possibly unreacted CO₂ and hydrogen and a liquid phase consisting of water.

Due to the exacerbated catalytic activity of the catalyst according to the invention, the inventors have noted that, surprisingly, the CO₂ conversion method can be carried out under specific operating conditions in a mode called “auto-methanation” mode, wherein the heat released within the catalytic bed is sufficient to maintain the reaction without the need for an external energy supply.

This “auto-methanation” mode of operation is possible when a reactor is used with a fixed catalyst bed, with a gas hourly space velocity of at least 15 m³/kg/h in the presence of a gas mixture having an H₂/CO₂ molar ratio comprised between 3 and 4, preferably equal to 4, and with a temperature within the catalytic bed comprised between 200° C. and 450° C., preferably between 220° C. and 300° C., after stabilisation of the heat flows in the catalytic bed.

In order to limit the risks of thermal runaway in the reactor, several measures can be taken alone or in combination. Thus it can be chosen to:
i) work on the dilution of the reaction flow: when starting the reaction, a more diluted flow (by adding an inert gas) could be used in order to reduce the heat released by the reaction, then, when the optimal temperature of the reaction is reached, the concentration of the reagents is increased step by step in order to promote the conversion of the reagents while maintaining an equilibrium between heat released by the reaction and the heat evacuated so as not to cause a sudden thermal runaway that is difficult to control;
ii) replace Joule effect heating with high thermal inertia in terms of regulation with inductive heating as described above;
iii) apply forced cooling of the reactor by means of circulation of a cooling fluid to remove heat and thus maintain the reaction temperature.

### BRIEF DESCRIPTION OF THE FIGURES

FIG. 1A schematically illustrates the assembly used by the inventors to carry out the catalytic tests under indirect Joule heating.

FIG. 1B schematically illustrates the assembly allowing induction heating of the catalytic bed.

FIGS. 2A to 2C respectively represent the methane yield as a function of the oven temperature, denoted T_{F}, the variation in the temperature of the catalytic bed as a function of the oven temperature and the methane yield as a function of the catalyst bed temperature obtained during tests with a Ni10/UO₂₊ₓ catalyst in the presence of a gas mixture with a H₂/CO₂ molar ratio of 4, with a gas hourly space velocity of 10 L/g/h.

FIGS. 3A to 3D represent the variation in the temperature of the catalytic bed as a function of the oven temperature obtained during tests with a Ni10/UO₂₊ₓ catalyst in the presence of a gas mixture with a H₂/CO₂ molar ratio of 4, for a gas hourly space velocity of 15, 20, 25 and 30 L/g/h.

FIGS. 4A to 4D represent the methane yield as a function of the temperature of the catalytic bed obtained during tests with a Ni10/UO₂₊ₓ catalyst in the presence of a gas mixture with an H₂/CO₂ molar ratio of 4, for a gas hourly space velocity of 15, 20, 25 and 30 L/g/h.

FIGS. 5A to 5C respectively represent the methane yield as a function of the oven temperature, the variation of the temperature of the catalytic bed as a function of the oven temperature and the methane yield as a function of the temperature of the catalytic bed obtained during tests with the Ni10/UO₂₊ₓ and Ni10/Al₂O₃ catalysts in the presence of a gas mixture with a H₂/CO₂ molar ratio of 4, with a gas hourly space velocity of 20 L/g/h.

FIGS. 6A to 6C show the methane yield as a function of the temperature of the catalytic bed obtained during tests with a Ni20/UO₂₊ₓ catalyst in the presence of a gas mixture with an H₂/CO₂ molar ratio of 4, with a gas hourly space velocity of 20 L/g/h.

FIG. 7 illustrates the methane yield as a function of time for a catalytic test carried out with a Ni20/UO₂₊ₓ catalyst, in the presence of a gas mixture with an H₂/CO₂ molar ratio of 4 and with a gas hourly space velocity of 20 L/g/h, wherein the catalytic bed was heated by induction using a coil.

FIG. 8 illustrates the functional yield during a conversion of CO₂ into methane in “auto-methanation” mode in the presence of a Ni20/UO₂₊ₓ catalyst with a gas mixture with a H₂/CO₂ molar ratio of 4, under a gas hourly space velocity of 20 L/g/h, wherein the catalytic bed was previously heated by induction by means of a coil.

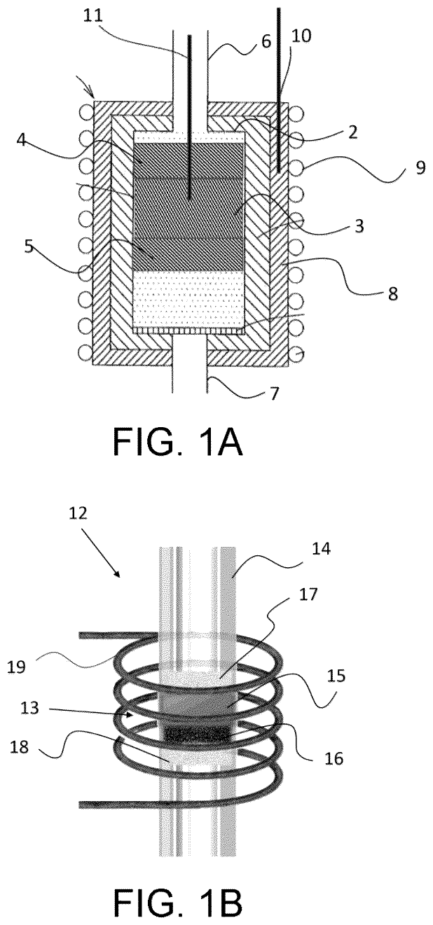

FIG. 1A is a representation of the micropilot device used for the study of the catalytic CO₂ conversion reaction. The device 1 includes a glass enclosure 2 with an internal diameter DI of 6 mm containing a catalyst bed 3 between an upper layer 4 and a lower layer 5 of quartz wool. The enclosure 2, comprising an inlet 6 for the reactive gases and an outlet 7 for the effluent, is received in an oven 8 equipped with heating structures 9 surrounding the wall of the oven.

The device is also equipped with a first thermocouple 10 placed in the wall of the oven and a second thermocouple 11 immersed in the catalytic bed allowing to follow respectively the temperature profile of the oven T_{F} and that of the catalytic bed T_{C} during the tests.

The micropilot is powered by gas cylinders: hydrogen, carbon dioxide and argon as purge gas. Gas flowrates are measured and regulated by mass flow meters coupled with solenoid valves. The temperature of the incoming gases is adjusted by passing through a preheater, the temperature of which can be regulated up to 400° C., before feeding the catalytic reactor. The operating mode described here is intended for a methanation reaction under indirect Joule heating.

The gas effluent recovered at the outlet of the reactor enclosure is cooled via a glass condenser then a Peltier effect condenser (T≈10° C. The water is therefore considered to be completely condensed when the output gas effluent is analysed.

The operating pressure in the micropilot is regulated by a regulation valve located downstream of the condenser.

The dry gas effluent is thus sampled downstream of the regulation valve and analysed using a gas phase micro-chromatograph (R3000, SRA Instrument) which is equipped with two different columns: a molecular sieve column (MS5A) allowing the separation of CO, CH₄ and H₂ and a polymer adsorbent column (PPU) allowing, inter alia, the analysis of CO₂, methane, ethane, ethylene and acetylene.

To carry out catalytic conversion tests, hydrogen is always introduced into the reactor before carbon dioxide. When changing the reaction temperature, the operating conditions are maintained until the temperature within the reactor and the composition of the gas mixture leaving the reactor have stabilised. The time required for this stabilisation is approximately 40 minutes. From the steady state of the operating conditions, the catalytic reaction is then maintained for at least one hour.

At the end of the tests, the reactor temperature is lowered to room temperature under a flow of argon before discharging the catalyst. The analysis of the composition of the dry gas and the measurement of its flowrate allow to calculate the CO₂ conversion rate and the CH₄ selectivity obtained.

The CO₂ conversion rate is defined as follows:

$${X}_{CO 2} = ( 1 - \frac{{Flowrate}_{output} - {A}_{CO 2 , output}}{{Flowrate}_{input} * {A}_{CO 2 , inpout}} ) * 100$$

with:
A_{CO2,input}≈area of the CO₂ peak of the gas flow entering the reactor measured by the micro-chromatograph;
A_{CO2,output}≈area of the CO₂ peak of the gas effluent leaving the reactor measured by the micro-chromatograph;
Output flowrate=flowrate of the gas effluent leaving the reactor;
Input flowrate=flowrate of the gas flow (H₂+CO₂) entering the reactor.

When the methane selectivity is equal to 100%, the CO₂ conversion rate can be calculated according to the following equation:

$${X}_{CO 2} = {X}_{CH 4} = \frac{{x}_{CH 4 , output}}{{x}_{CH 4 , output} + {x}_{CO 2 , output}} * 100 ,$$

X_{CH4,output} being the molar concentration of methane in the effluent.

The above relationship can also be written, considering that the water formed by the reaction is completely condensed in the output condenser and that there is no formation of CO:

$${X}_{CH 4} = {X}_{CO 2} = \frac{( 1 + R ) \cdot {x}_{CH 4 , output}}{( 1 + 4 ) \cdot {x}_{CH 4 , output}} * 100 ,$$

R being the H₂/CO₂ molar ratio in the reaction mixture.

The last equation uses only one experimental parameter x_{CH4,output} which is the molar concentration of methane formed and the relative uncertainty of which is the lowest (less than 5%).

To the extent that the methane selectivity is of 100%, the methane yield of the reaction is equal to the CO₂ conversion rate determined by the formulas above.

The catalysts according to the invention were prepared from a U₃O₈ powder (Prolabo) which is shaped by spheronisation in the presence of distilled water.

The beads obtained are then impregnated with an aqueous solution of nickel nitrate using the dry impregnation technique. The total pore volume of the U₃O₈ beads was determined by dry impregnation with a distilled water solution.

The incorporation of nickel on the support by impregnation from an aqueous solution of nickel nitrate was carried out in a single step to provide catalysts the mass content of which is 10%, 15% and 20% of nickel relative to the total mass of catalyst.

At the end of the impregnation step(s), the solid is dried in air for 3 hours then calcined in air in a sealed tubular reactor at 350° C. for 2 hours.

The calcined beads are sieved in order to recover only the fraction having a grain size between 0.2 mm and 0.8 mm.

Before being used in the conversion reaction, the calcined beads are activated by reduction under a flow of pure hydrogen (50 mL/min) at 350° C. for two hours.

X-ray diffraction analysis of the catalysts after reduction under hydrogen indicates the presence of a majority UO₂ phase, a minority U₃O₈ and nickel metal.

### EXAMPLES

In the examples which follow, the hourly space velocities are expressed in relation to the amount of catalyst used.

### Example 1: Evaluation of the Ni10UO₂₊ₓ Catalyst at a Gas Hourly Space Velocity of 10 L/g/h Under Indirect Joule Heating

The catalytic activity of the Ni catalyst (10% by weight) on a UO₂₊ₓ support prepared according to the method described above.

400 mg of catalyst was previously mixed with silicon carbide (SIC) as an inert diluent. The mixture is introduced into the glass reactor and forms a catalytic bed approximately 12 mm thick.

Tests were carried out with a gas mixture, the H₂/CO₂ ratio (mol/mol) of which is equal to 4 for a gas hourly space velocity of 10 L/g/h, at a pressure of 0.1 MPa and by following the oven temperature T_{F} and that of the catalytic bed T_{C}.

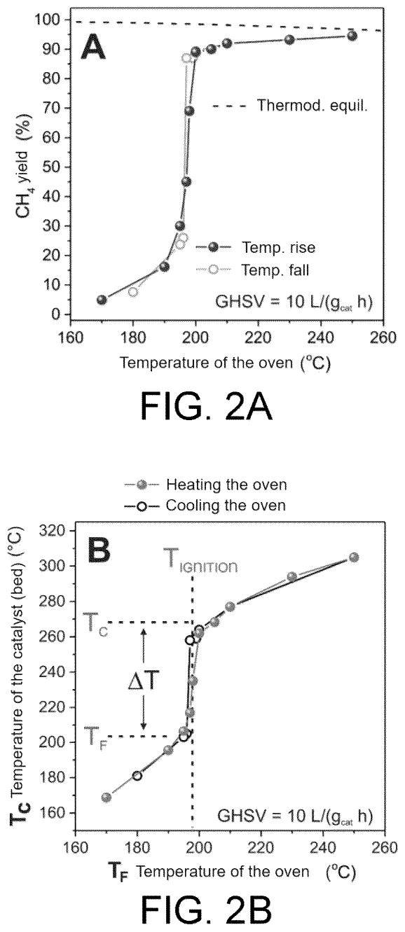

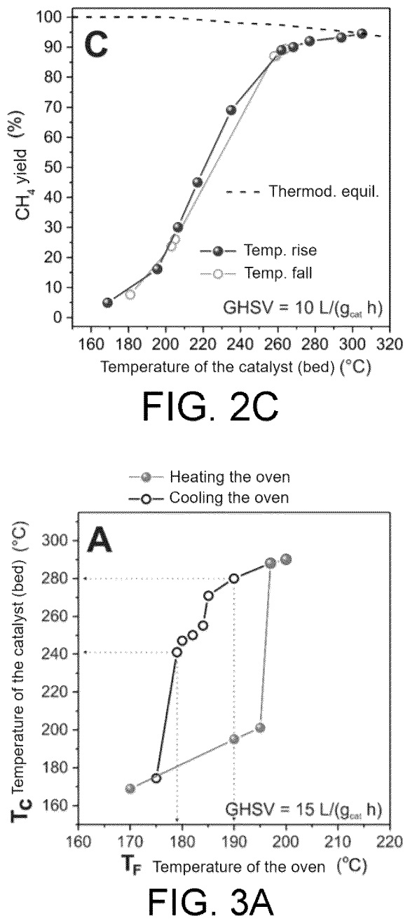

With reference to FIG. 2A, it is seen that the conversion of CO₂ remains low for oven temperatures T_{F} lower than 197° C. Beyond this value, the catalytic reaction starts suddenly and the determined CO₂ conversion is approximately 90% for a temperature T_{F} of 200° C. Above 200° C., the CO₂ conversion rate reaches an asymptote which tends towards the value of 95% for a temperature above 200° C., close to thermodynamic equilibrium.

With reference to FIG. 2B which represents the variation of the temperature in the catalytic bed T_{C} as a function of the oven temperature T_{F}, it is noted that when the oven temperature T_{F} reaches 197° C., the measured temperature of the catalytic bed T_{C} is of the order of 270° C. This temperature difference ΔT (T_{C}-T_{F}) of approximately 70° C. corresponds to the heat released by the exothermicity of the reaction which cannot be evacuated from the catalytic bed by exchanges with the gas flow. This temperature difference allows to understand the CO₂ conversion jump observed in FIG. 2A.

Monitoring of the catalytic reaction was also carried out during the reactor cooling process by measuring the conversion of CO₂ as a function of the temperature in the catalytic bed T_{C}. Cooling was carried out by reducing the setpoint oven temperature, while maintaining the injection of the H₂ and CO₂ gas flow. The objective is to determine if a hysteresis in the catalytic process is observable when reducing the oven temperature T_{F}, which would result in maintaining the conversion of CO₂ thanks only to the heat released by the reaction.

With reference to FIG. 2C which gives the CH₄ yield as a function of the temperature measured in the catalytic bed T_{C} during the rise and fall in temperature in the catalytic bed, it is seen that the exothermic reaction releases strong heat in the catalytic bed. However, under these reaction conditions, the heat released within the catalytic bed is insufficient to achieve a state of equilibrium between the heat produced by the reaction and that which is evacuated by the gas effluent leaving the reactor. Indeed, during the slow descent of the oven setpoint temperature T_{F}, the CO₂ conversion falls with the temperature T_{C}. No catalytic hysteresis is observed under the operating conditions of Example 1.

### Example 2: Evaluation of the Ni10/UO₂₊ₓ Catalyst at Gas Hourly Space Velocities of 15, 25 and 30 L/g/h Under Indirect Joule Heating

The tests of Example 2 were carried out with the same reactor configuration as that of Example 1 in order to study the catalytic behaviour of the catalyst containing 10% by mass of Ni relative to the total mass of catalyst, at higher gas hourly space velocities during the phases of rise and fall in oven temperature. These tests allow in particular to determine whether hysteresis (conversion deviation during the rise and fall of temperature) can be observed.

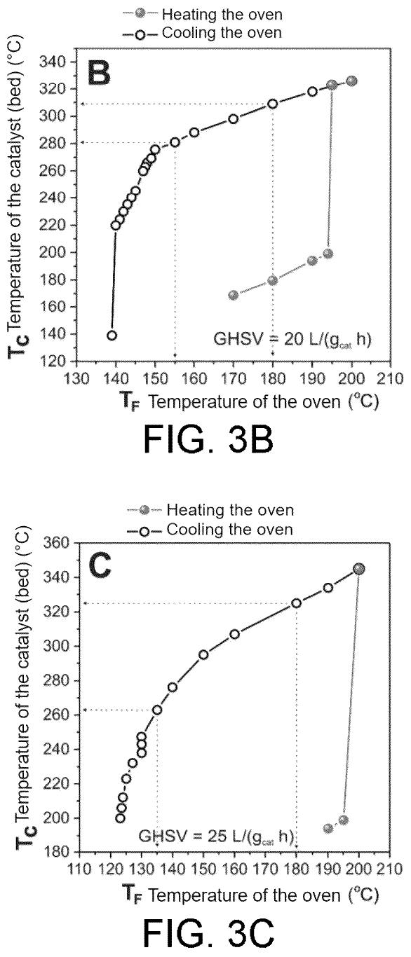

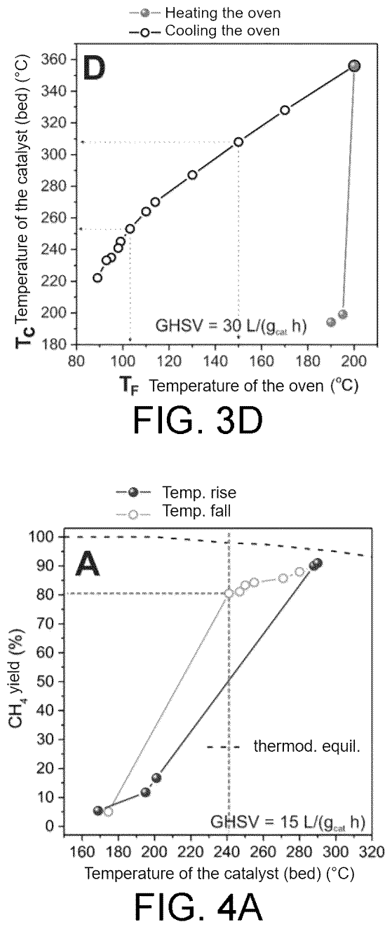

FIGS. 3A to 3D represent the evolution of the temperature of the catalytic bed T_{C} as a function of the oven temperature T_{F} during the phase of rise and fall in oven temperature for the different values of gas hourly space velocity equal to 15, 20, 25 and 30 L/g/h, in the presence of a gas flow containing a H₂/CO₂ molar ratio equal to 4.

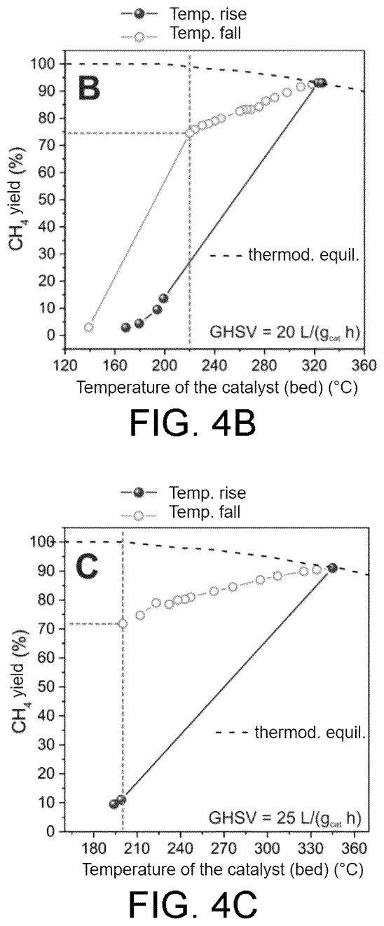

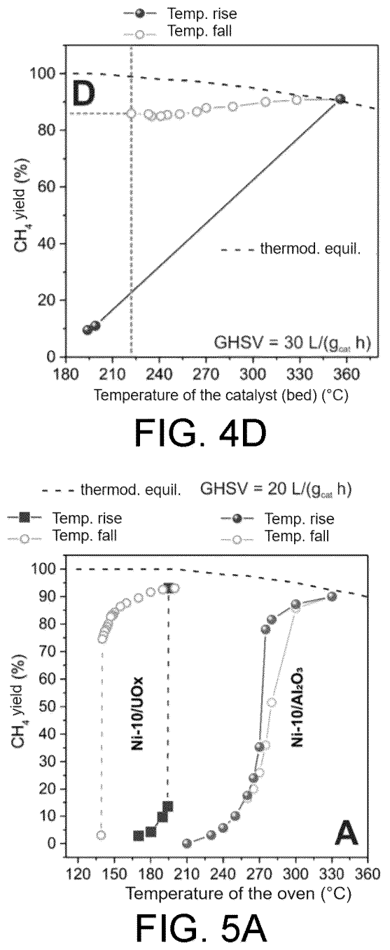

As for FIGS. 4A to 4D, they indicate the methane yield, representative of the methanation activity of the catalyst, as a function of the temperature of the catalytic bed and at the different gas hourly space velocities during the phases of rise and fall in oven temperature.

With the Ni10/UO₂₊ₓ catalyst and for a gas hourly space velocity greater than or equal to 15 L/g/h, hysteresis appears when the oven temperature decreases. This phenomenon is all the more notable as the gas hourly space velocities are high (FIGS. 3A to 3D). Indeed, the methanation activity gradually decreases but still remains high for temperatures measured in the catalytic bed above 200° C.

It is further observed that the maximum temperature measured in the catalytic bed T_{C} increases with the hourly space velocity and that the fall in temperature in the catalytic bed during the fall in oven temperature is even less rapid as the gas hourly space velocity is high. This therefore reflects that the heat generated by the high conversion of CO₂ allows to maintain the temperature of the catalytic bed (FIGS. 4A to 4D).

In the case of the test carried out at a gas hourly space velocity of 30 L/g/h (FIG. 3D), it was noted that, even when the oven was turned off, the temperature displayed by the oven thermocouple remained above 90° C., indicating that part of the heat generated in the catalytic bed (the temperature of which is maintained at approximately 220° C. thanks to the exothermicity of the reaction) contributes to maintaining the oven temperature thanks to the heat exchanges.

The catalytic system according to the invention is capable of operating in “auto-methanation” mode when the gas hourly space velocity is at least 25 L/g/h (that is to say 25 m³/kg/h) and as long as the temperature of the catalytic bed remains at a value greater than or equal to 200° C., thanks to the establishment of an equilibrium between the heat released by the reaction and the heat exchanged with the flow of reagents.

### Example 3 (comparative): Evaluation of the Ni10/gamma-Alumina Catalyst Compared to the Ni10/UO₂₊ₓ Catalyst at 20 L/g/h Under Indirect Joule Heating

Comparative tests were carried out under the same operating conditions as those of Example 1, that is to say with a gas mixture the H₂/CO₂ molar ratio of which is 4 but in the presence of a catalyst comprising a mass content of nickel on an alumina type support (Al₂O₃).

The comparative catalyst was prepared as follows: the support based on gamma-Al₂O₃, subsequently denoted alumina, is in the form of extrudates (1 mm in diameter and 3 mm in length, Ketjen 300B supplied by Akzo Nobel) is dry impregnated with an aqueous solution of nickel nitrate at room temperature. The material is left to mature at room temperature for 3 hours then dried at 110° C. in air for 3 hours in order to eliminate the solvent. The dry material is calcined in air at 350° C. for 2 hours (with a temperature rise slope of 3° C./min). The catalyst precursor thus obtained is reduced directly in the catalytic reactor under a flow of pure hydrogen (20 mL/min) at 350° C. for 2 hours.

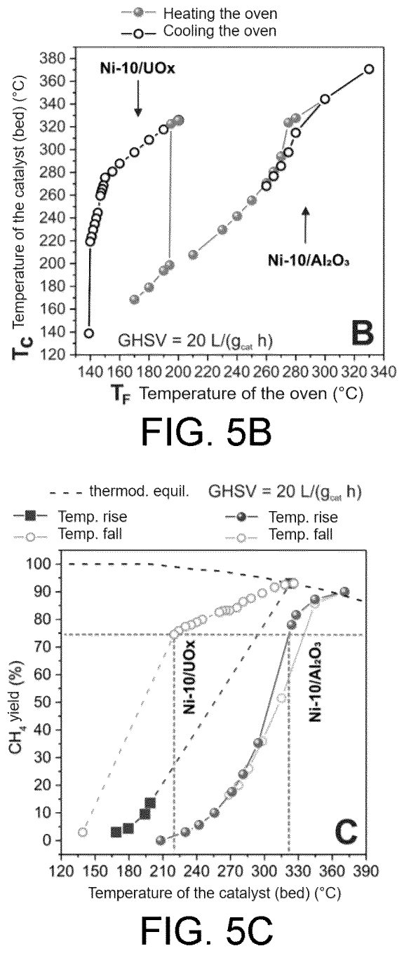

The experimental results are reported in FIGS. 5A and 5C which give the methane yield as a function of the oven temperature T_{F} and the catalytic bed Tc during the phases of rise and fall in oven temperature. FIG. 5A confirms the excellent methanation activity of the Ni10/UO₂₊ₓ catalyst compared to its Ni10/Al₂O₃ counterpart.

FIG. 5B shows the evolution of the temperature in the catalytic bed To as a function of the oven temperature T_{F} during the phases of rise and fall in oven temperature. For the Ni10/UO₂₊ₓ catalyst, when the oven temperature exceeds 190° C., a significant jump in the temperature in the catalyst bed is measured (320° C. This jump is explained by a higher conversion of CO₂ into CH₄. In the case of the Ni10/Al₂O₃ catalyst, a very small difference between the two temperatures, which reflects a lower conversion is observed.

With reference to FIG. 5C, it is seen that at iso-yield in CH₄, the temperature in the Ni10/Al₂O₃ catalytic bed is always higher than that measured in the Ni10/UO₂₊ₓ catalytic bed. For example, as indicated in FIG. 5C, for a CH₄ yield of 75%, the temperature measured in the catalytic bed containing Ni10/Al₂O₃ catalyst is 320° C. while that measured in the catalytic bed containing the Ni10/UO₂ catalyst is 220° C. These results therefore confirm the strong reactivity of the Ni10/UO₂₊ₓ catalyst.

The slightest catalytic activity of the Ni10/Al₂O₃ catalyst is also manifested by the fact that the catalytic activity is not maintained when the oven temperature falls and therefore by the absence of hysteresis in the temperature profile.

### Example 4: Evaluation of the Ni20/UO₂₊ₓ Catalyst at Gas Hourly Space Velocities of 20 L/g/h and 30 L/g/h Under Indirect Joule Heating

Tests were carried out with a catalyst comprising 20% by mass of nickel relative to the total mass of catalyst on a UO2+x support. This catalyst was prepared according to the same protocol as that of Example 1 with a double dry impregnation of an aqueous solution of nickel nitrate.

The catalyst after reduction under hydrogen was tested under two gas hourly space velocities of 20 L/g/h and 30 L/g/h and with a gas mixture with a H₂/CO₂ molar ratio of 4.

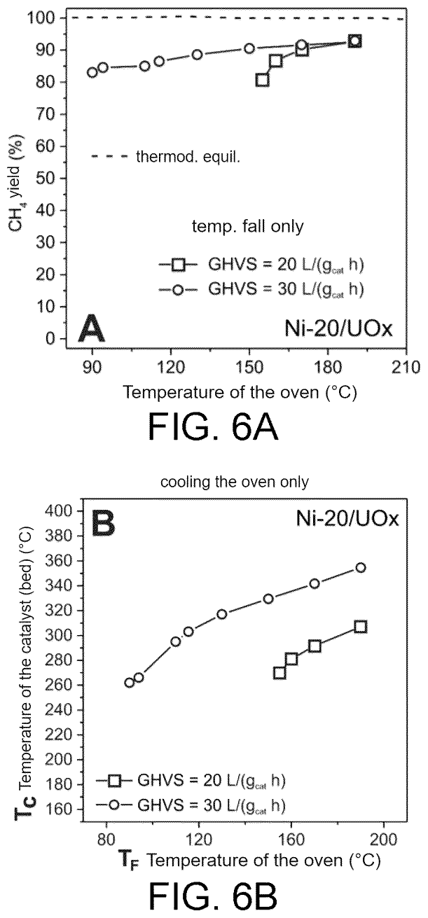

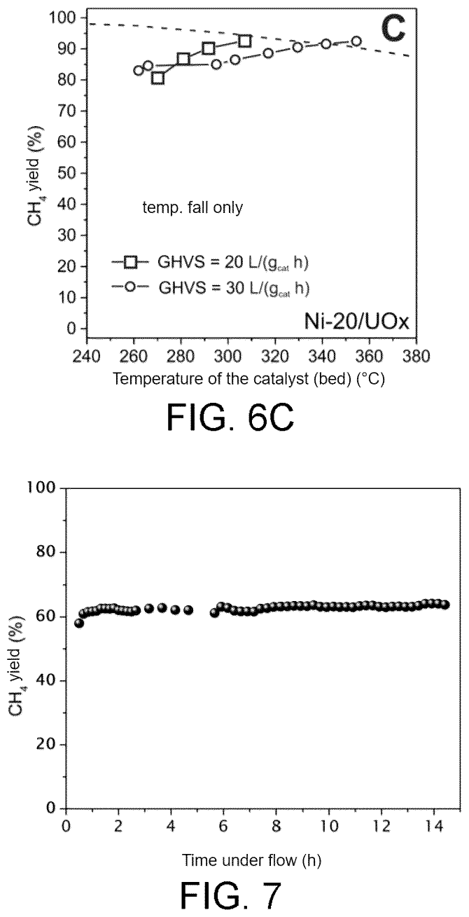

FIGS. 6A to 6C show respectively, for the phase of fall in oven temperature, the CH₄ yield as a function of the oven temperature T_{F}, the variation in the temperature of the catalytic bed as a function of the oven temperature and finally the CH₄ yield as a function of the temperature of the catalytic bed T_{C}.

It can be observed that the catalyst according to the invention containing 20% by mass of Ni has excellent catalytic activity. The CH₄ yield remains greater than 80% for a temperature range in the catalytic bed comprised between 260° C. and 360° C. With reference to FIGS. 6A, 6B and 6C, it is also noted that the catalyst is capable of operating in the auto-methanation mode during the fall in oven temperature. For example, for a gas hourly space velocity of 30 L/g/h and a temperature of 90° C. measured in the oven, the CH₄ yield remains greater than 80% thanks to maintaining the temperature of the catalytic bed at a value of approximately 260° C.

### Example 5: Evaluation of the Ni20/UO₂₊ₓ Catalyst at Low Temperature and Gas Hourly Space Velocities of 20 L/g/h Under Inductive Heating

Tests were carried out with a catalyst comprising 20% by mass of nickel relative to the total mass of the catalyst on a UO₂₊ₓ support.

The device 12 used to carry out the heating is shown in FIG. 1B.

The catalytic bed 13, which is contained in a glass reactor 14 with an internal diameter of 8 mm, includes a layer 15 consisting of 800 mg of catalyst mixed with 200 mg of silicon carbide as thermal diluent (SiC). The catalyst and SiC mixture is deposited above a graphite felt 16 acting as a susceptor. The thickness of the catalytic bed 13, which is comprised between two layers of quartz wool 17, 18, measures approximately 17 mm.

The reactor 14 is heated by means of an EasyHeat 8310, 10 KW induction heating system marketed by the company Ambrell Ltd. This system is equipped with a six-turn copper induction coil 19 which is cooled by water circulating inside the coil. The reactor is disposed inside the coil so that the catalytic bed is surrounded by the induction coil. Real-time temperature control is provided by a Eurotherm 3504 regulator connected to an Optris™ laser pyrometer pointing at the catalytic bed.

After an in situ reduction step of the catalyst at 300° C. for 30 min under H₂, the latter is allowed to cool to 160° C. under Ar. The reaction mixture (1 mol of CO₂ for 4 mol of H₂) is introduced in the reactor with a gas hourly space velocity of 20 L/g/h.

The catalytic bed is gradually heated by induction to a temperature of 190° C. measured by the laser pyrometer. During the test, the temperature in the catalytic bed is set at the setpoint temperature of 190° C.

The results are presented in FIG. 7. It can be seen that the Ni20/UO_{2'x} catalyst has a stable CH₄ yield of approximately 62%, for a temperature in the catalytic bed of 190° C. These results indicate that the Ni20/UO₂ catalyst allows to carry out the methanation reaction at a relatively low temperature compared to the results reported in the literature. In addition, induction specifically heats only the solid catalyst and the susceptor and not the gas reagents passing through the catalytic bed, hence a significant gain in terms of energy input to the method. It should be noted that the catalyst does not have any deactivation during the duration of the test, thus confirming the excellent stability of the latter.

### Example 6: Evaluation of the Ni20/UO₂₊ₓ Catalyst at Gas Hourly Space Velocities of 20 L/g/h Under Inductive Heating

Tests were carried out with the same catalyst and the same reactor configuration as those of Example 5.

### Example 6 differs from Example 5 by the conditions for heating the catalytic bed.

The catalytic bed is heated by induction gradually to the setpoint temperature of 170° C. with a current applied to the inductor of 120 amps. Then the setpoint temperature is suddenly increased to 190° C. which is accompanied by a temperature jump within the bed which reaches 230° C. This exothermicity in the catalytic bed reflects a runaway of the catalytic CO₂ conversion reaction. The current applied to the inductor is then reduced to 23 amps then completely cut off and finally the induction coil is moved so that the catalytic bed is outside its environment.

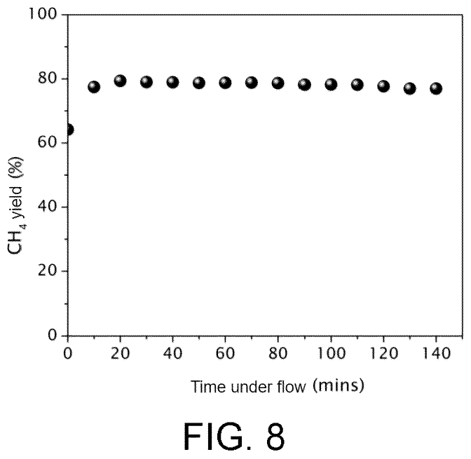

The results of the conversion of CO₂ into methane after stopping the inductor are presented in FIG. 8. The catalyst according to the invention achieves a conversion of approximately 80% of CO₂ into CH₄ in auto-methanation mode (without external heat supply) for 140 min. During this test, it was also noted that the temperature measured in the catalytic bed oscillates between 220° C. and 230° C. thanks to the heat released by the catalytic reaction.

What is claimed is:

1. A method for converting CO₂ into methane, wherein:
hydrogen is contacted with a gas feed comprising CO₂ in at least one methanation reactor comprising a catalyst bed, at a temperature in the catalyst bed comprised between 160° C. and 550° C., at a pressure comprised between 0.1 MPa and 1 MPa, with a gas hourly space velocity comprised between 10 m³/kg/h and 50 m³/kg/h and with an H₂/CO₂ molar ratio comprised between 1 and 8;
the catalyst comprises nickel metal deposited on a uranium oxide support of formula UO₂₊ₓ, with x being between 0.01 and 0.6, the nickel metal mass content of the catalyst being between 5% and 40% relative to the total mass of the catalyst; and wherein:
the catalyst is prepared by a method comprising the following steps a) to c):
a) a support precursor consisting essentially of a uranium (IV) oxide, a uranium (VI) oxide or a mixture thereof is impregnated with a solution comprising a nickel precursor and a polar solvent;
b) the impregnated support precursor is calcined in air and at a temperature of at least 250° C.; and
c) the impregnated and calcined support precursor is reduced under hydrogen at a temperature of at least 300° C.

2. The method of claim 1, wherein the impregnated support precursor is dried at a temperature below 200° C. before being calcined.

3. The method of claim 1, wherein the support precursor is U₃O₈, UO₂, UO₄ or UO₃.

4. The method of claim 1, wherein the support precursor is in the form of a powder, cylindrical extrudates, multi-lobed extrudates, spheres, rings, or monoliths with a honeycomb structure.

5. The method of claim to 1, wherein the nickel precursor is nickel hydroxide, hydroxycarbonate, nickel carbonate or nickel nitrate.

6. The method of claim 1, wherein the impregnation step a) comprises a dry impregnation or an excess impregnation of the support precursor.

7. The method of claim 1, wherein step c) is carried out in the methanation reactor.

8. The method of claim 1, wherein hydrogen and the gas feed comprising CO₂are sent separately or as a mixture into the methanation reactor in a downward direction.

9. The method of claim 1, wherein the methanation reactor is an adiabatic reactor.

10. The method of claim 1, wherein the methanation reactor comprises a fixed or fluidised catalyst bed.

11. The method of claim 10, wherein the catalyst bed is a fixed catalyst bed and is subjected to an alternating electromagnetic field so as to be heated by induction.

12. The method of claim 11, wherein the fixed catalyst bed further comprises susceptor particles.

13. The method of claim 1, wherein the method is carried out with a renewable energy source.

14. The method of claim 1, wherein the reactor comprises a fixed catalyst bed and wherein the gas hourly space velocity is fixed at least at 15 m³/kg/h for maintaining the temperature of the catalyst bed at least at 200° C., whereby no external heat is added.

15. A method for preparing a CO₂ methanation catalyst comprising nickel metal deposited on a uranium oxide support of formula UO₂₊ₓ with x being between 0.01 and 0.6, and wherein the mass content of nickel metal is between 5% and 40% relative to the total mass of the catalyst, which method comprises the following steps a) to c):
a) a support precursor consisting essentially of a uranium (IV) and/or uranium (VI) oxide is impregnated with a solution comprising a nickel precursor and a polar solvent;
b) the impregnated support precursor is calcined in air and at a temperature of at least 250° C.; and
c) the impregnated and calcined support precursor is reduced under hydrogen at a temperature of at least 300° C.
